(19) United States
(12) Patent Application Publication
(10) Pub. No.: US 2011/0269837 A1
(43) Pub. Date: Nov. 3, 2011

(54) PHOSPHORUS BINDER COMPOSITION FOR TREATMENT OF HYPERPHOSPHATEMIA

(75) Inventor: Deanna J. Nelson, Raleigh, NC (US)

(73) Assignee: BIOLINK LIFE SCIENCES, INC., Cary, NC (US)

(21) Appl. No.: 13/097,141

(22) Filed: Apr. 29, 2011

Related U.S. Application Data
(60) Provisional application No. 61/330,610, filed on May 3, 2010.

(51) Int. Cl. A61K 31/194; A61P 3/00; A61P 1/00; C07C 55/10

(52) U.S. Cl. 514/574; 562/590

(57) ABSTRACT

The present invention relates to oral pharmaceutical products which are useful for binding phosphorus in ingesta, and inhibiting absorption of phosphorus from the gastrointestinal tract of subjects. A method for binding phosphorus in ingesta and inhibiting its absorption from the gastrointestinal tract is also provided. The pharmaceutical products and methods of the present invention are particularly useful in the treatment of hyperphosphatemia of chronic uremia and reducing serum phosphorus levels in patients requiring such therapy.

### CROSS-REFERENCE TO RELATED APPLICATION

This application claims the benefit of and priority to pending U.S. Provisional Patent Application Ser. No. 61/330,610, filed May 3, 2010, the entire contents of which are hereby incorporated by reference in its entirety.

### FIELD OF THE INVENTION

The present invention relates to oral pharmaceutical products which are useful for binding phosphorus in ingesta, and inhibiting absorption of phosphorus from the gastrointestinal tract. A method for binding phosphorus in ingesta and inhibiting its absorption from the gastrointestinal tract is also provided. The pharmaceutical products and methods of the present invention are useful in the treatment of hyperphosphatemia and reducing serum phosphorus levels in patients requiring such therapy.

### BACKGROUND OF THE INVENTION

In the adult human, normal serum phosphorus levels range from 2.5 to 4.5 mg/dL (0.81 to 1.45 mmol phosphorus/L). (Normal serum levels are typically 50% higher in infants and 30% higher in children due to growth hormone effects.) Hyperphosphatemia is a disease state in which there is an abnormally elevated serum phosphorus (Pi) level in the serum. Significant hyperphosphatemia is considered present when serum phosphorus levels are greater than about 5 mg/dL in adults or 7 mg/dL in children or adolescents. [National Kidney Foundation. Am J Kidney Dis 2003; 42 (Suppl 3):S1-S201.]

The kidney plays a central role in maintaining a condition of phosphorus homeostasis in the body of a subject, wherein the amount of phosphorus absorbed from the gastrointestinal tract approximately equals the amount excreted via the kidney. In addition, cellular release of phosphorus is balanced by uptake in other tissues. Hormonal control is provided by parathyroid hormone.

Since the kidney plays a central role in maintaining phosphorus homeostasis, kidney dysfunction is often accompanied by increased phosphorus retention by the body. In early kidney dysfunction, compensatory physiological responses allow for a continued match between urinary phosphorus excretion and phosphorus absorption from the gastrointestinal (GI) tract, and serum phosphorus levels remain near normal values. With more advanced renal failure, however, elevated serum phosphorus is a predictable co-morbidity.

In patients with chronic kidney disease (CKD), phosphorus retention (as evidenced by abnormally elevated serum phosphorus levels) may contribute to progression of renal failure and is a major factor in the development of secondary hyperparathyroidism, renal osteodystrophy, and soft tissue calcification. [Tonelli M, Pannu N, Manns B. Oral phosphate binders in patients with kidney failure. New England J Med 2010; 362: 1312-1324.] Hyperphosphatemia in CKD patients is treated by restriction of phosphorus in the diet and pharmacological means. Serum phosphorus is also reduced during dialysis of end-stage renal disease patients undergoing such treatment.

Phosphorus is present in nearly all foods, and absorption of dietary phosphorus from ingesta in the gastrointestinal (GI) tract is very efficient. Normal daily dietary intake varies from 800-1,500 mg of phosphorus. Typically, 70-90% of dietary phosphorus is absorbed, primarily from the jejunum, duodenum, and proximal ileum of the GI tract, although some absorption continues throughout the remainder of the intestinal tract. A small amount of GI excretion occurs.

The efficient absorption of phosphorus from food and the need to provide a diet sufficient to counter the catabolic state of CKD render dietary restriction insufficient to prevent hyperphosphatemia. Moreover, conventional dialysis fails to reduce levels of phosphorus in the blood, and serum phosphorus levels increase with time. Therefore, prevention of phosphorus absorption with pharmacological means is generally required to prevent or reverse hyperphosphatemia and the morbidities and mortality risks associated with it.

Methods for the prevention and treatment of hyperphosphatemia include the use of compounds or compositions that will bind phosphorus in the gastrointestinal (GI) tract of a subject and prevent its absorption into the systemic circulation. Compounds that bind phosphorus in the GI tract are known as phosphorus (Pi) binders. Today, oral phosphate binders are used in over 90% of patients with kidney failure and/or hyperphosphatemia, at an annual cost of approximately $750 million (U.S. dollars) worldwide.

Phosphorous binding is a chemical reaction between dietary phosphorus and a cation of a binder compound. Chemical reaction results in the formation of insoluble and hence unabsorbable phosphate compounds, adsorption of phosphorus-containing anions on the surface of binder particles, or a combination of both processes.

Metal salts comprise the most clinically important class of phosphorus binders and are ingested to bind dietary phosphate and convert it to insoluble phosphate salts, thus preventing its absorption from the GI tract. Known metal salts with phosphate-binding properties are aluminum hydroxide and aluminum carbonate; calcium acetate, calcium carbonate, calcium citrate, calcium alginate, calcium gluconate, calcium lactate, and calcium sulfate; lanthanum carbonate and lanthanum carbonate hydrates; magnesium carbonate and magnesium hydroxide; as well as complex salts of iron. (Not all of these salts have gained therapeutic importance or been considered safe or efficacious for Pi binding.) Polymeric materials having a plurality of cationic sites (e.g., tertiary and quaternary amines) appended thereto constitute the second clinically important class of phosphorus binders.

Phosphorus binding by metal salts is affected by the pH of the solution environment. The solution pH affects both the rate of dissolution of the metal salt and the subsequent binding reaction between the metal ion and phosphate. In general, an acidic pH is best to dissolve and ionize the salt, but reaction of the metal with the phosphate and precipitation of metal phosphate from solution is optimal at higher values of pH.

Of the metal salts listed above, calcium salts constitute the group of phosphate binders used most extensively by patients worldwide to control serum Pi levels (Table 1).

TABLE 1
Calcium salts that have been studied as Pi binders

| | % Ca, | |
|---|---|---|
| Calcium | by | |
| Source | weight | Remarks |
| Calcium | 40% | Wide variations in ionized Ca bioavailability and |
| Carbonate | | trace metal contamination; widely used outside the |
| | | U.S. for Pi binding |
| Calcium | 23% | Regurgitation of acetic acid (vinegar breath) is a |
| Acetate | | significant side effect. Only calcium acetate has |
| | | been approved by the U.S. Food and Drug |
| | | Administration for clinical use as a phosphate |
| | | binder. |
| Calcium | 21% | Enhances absorption of calcium and other metals |
| Citrate | | from the gut, including (adventitious) dietary |
| | | aluminum. |
| Calcium | 31% | Chronic absorption of formate is reported to cause |
| Formate | | albuminuria and hematuria. Pungent odor. Skin |
| | | irritant. |
| Calcium | 14% | Used as a dietary supplement, not as a Pi binder. |
| Lactate | | |
| Calcium | 9% | Used as a dietary supplement, not as a Pi binder. |
| Gluconate | | |

In U.S. Pat. No. 4,889,725 Veltman discloses a means for promoting the neutralization reaction between particulate calcium carbonate and ionized phosphate by adding a material formed by the reaction of particulate calcium carbonate and dilute hydrofluoric acid. The products of this invention are useful in lowering serum phosphorus levels in patients undergoing renal dialysis, and are also useful as antacids.

A common treatment for controlling Pi levels is disclosed in U.S. Pat. No. 4,870,105 to Fordtran, which discloses a calcium acetate phosphorus binder for oral administration to an individual for the purpose of inhibiting gastrointestinal absorption of phosphorus. It further discloses a method of inhibiting gastrointestinal absorption of phosphorus, comprising administering orally the calcium acetate phosphorus binder, preferably close in time to food and beverage consumption. Likewise, U.S. Pat. No. 6,576,665 to Dennett, Jr. et al. discloses a composition for inhibiting gastrointestinal absorption of phosphorus in an individual. The composition includes a quantity of calcium acetate sufficient to bind the phosphorus and having a bulk density of between 0.50 kg/L and 0.80 kg/L and is dimensioned to form a caplet for fitting within a capsule. Further provided is a method for administering the calcium acetate composition. Likewise, U.S. Patent Application 2003/0050340 to Dennett, Jr., et al. discloses a composition for binding phosphorus within the gastrointestinal tract of an individual. The composition includes a quantity of calcium acetate having a specific bulk density sufficient to bind the phosphorus in the gastrointestinal tract of an individual. Further provided is a method for administering the calcium acetate composition.

U.S. Pat. No. 4,689,322, to Kulbe et al. provides calcium salts or calcium mixed salts of polymeric, anionic carboxylic acids and/or an ester of sulfuric acid, and methods for their preparation and use, discloses a pharmaceutical product which contains at least a calcium salt or a calcium mixed salt of a natural or chemically modified polymeric, anionic carboxylic acid and/or an ester of sulfuric acid, and additive materials and/or carrier materials. There are further disclosed calcium salts, and methods of preparation thereof, comprised of polymannuronic acid, polygalacturonic acid, polyglucuronic acid, polyguluronic acid, the oxidation products of homoglycans, the oxidation products of heteroglycans, or their mixtures, for controlling the levels of phosphorus, calcium and iron in patients with chronic uremia and/or the control of the oxalate and/or phosphate of the blood in kidney stone prophylaxis.

U.S. Pat. Nos. 6,160,016 and 6,489,361B1 to DeLuca disclose a calcium formate composition for oral administration to an individual for the purpose of inhibiting gastrointestinal absorption of phosphorus. It further discloses a method of inhibiting gastrointestinal absorption of phosphorus, comprising administering orally the composition, preferably close in time to food and beverage consumption. Further, DeLuca discloses a method of inhibiting gastrointestinal absorption of phosphorus, comprising administering orally the calcium formate composition of his invention, preferably close in time to food and beverage consumption.

U.S. Pat. No. 6,887,897 B2 (Walsdorf et al.) discloses a calcium glutarate supplement and its use for controlling phosphate retention in patients on dialysis and suffering from renal failure and associate hyperphosphatemia. Therapeutic benefit can be realized by administering a calcium glutarate compound orally to a patient to increase available calcium and contact and bind with ingested phosphorus in the patient's digestive tract, and thereby prevent its intestinal absorption.

In U.S. Pat. No. 6,926,912 B1 Roberts et al. disclose a non-aluminum containing mixed metal compound or sulphated metal compound useful as phosphate binders in the treatment of hyperphosphatemia. The mixed metal compounds include a mixed metal hydroxyl carbonate containing magnesium and iron and may have a hydrocalcite structure, preferably a non-aged hydrocalcite structure. The phosphate binders disclosed by Roberts et al. have a phosphate binding capacity of at least 30% by weight, based on test methods described in the specification.

In U.S. Pat. No. 7,517,402 B2 Muhammad discloses a phosphate binder, a composition, and a kit, as well as a process for preparing the binder and composition. The binder is characterized as having calcium silicate sites which are connected the one with the other by alumina-silica phosphate bonds.

Ingestion of each of the conventional calcium-containing phosphate binders listed above causes the subject to experience significant side effects. Ingestion of calcium carbonate, for example, causes side effects that include distaste, nausea, flatulence, and constipation. Similarly, ingestion of calcium acetate causes side effects that include distaste, nausea, regurgitation of acetic acid, and constipation. Ingestion of calcium formate causes side effects that include distaste, nausea, albuminuria, and constipation. Ingestion of mixed metal or sulphated compounds having calcium silicate sites which are connected by alumina-silica bonds causes side effects that include absorption of aluminum from the composition and constipation.

The ideal Pi binder should bind most dietary phosphorus in the gastrointestinal tract without producing significant side effects. It should also be relatively inexpensive, because most patients having or at risk for hyperphosphatemia consume relatively large daily doses of a gram or more of the binder. Unfortunately, none of the conventional Pi binders fulfill all of these requirements. It would be very useful, therefore to have a Pi binder which binds dietary phosphorus more effectively, thus enabling use of lower doses, and which does not have the risks and side effects associated with ingestion of conventional calcium salt Pi binders. The present invention answers this unmet need.

### SUMMARY OF THE INVENTION

The present invention is a pharmaceutical composition for use in the treatment of hyperphosphatemia comprising a phosphorus-binding composition. A method of inhibiting phosphorus absorption from the gastrointestinal tract is provided, comprising administering a phosphorus-binding composition. A method of treating hyperphosphatemia in a warm-blooded animal with a therapeutically effective amount of a phosphorus-binding composition is disclosed. A pharmaceutical composition useful for treating hyperphosphatemia in a warm-blooded animal and reducing the risk of side effects is also disclosed. A method of reducing serum phosphorus levels in a warm-blooded animal is disclosed, comprising treating the animal with a therapeutically effective amount of a phosphorus-binding composition that is administered orally, whereby phosphorus absorption from the gastrointestinal tract is inhibited.

### DESCRIPTION OF THE DRAWINGS

FIG. 1 is a graph comparing the percentage of phosphate bound by a phosphate binder of the invention and a conventional phosphate binder, calcium acetate, in the pH range from pH 4 (the pH of the stomach with food present) to pH 8 (the pH of the lower intestine).

FIG. 2 is a graph comparing the percentage of phosphate bound by a phosphate binder of the invention comprising a calcium succinate composition in the absence or the presence of a stool softener (Binding Solution 1 and Binding Solution 2, respectively). Phosphate was present in excess in the solutions.

### DETAILED DESCRIPTION OF THE INVENTION

The present invention relates to a phosphorus-binding composition for oral administration to a subject. The composition is useful for reducing phosphorus absorption from the GI tract of a subject and reducing the risk of side effects of the treatment.

The present invention also relates to a method of inhibiting phosphorus absorption from the gastrointestinal tract and reducing the risk of side effects of the treatment. The method of the present invention is based on the demonstration that a phosphorus-binding composition comprising a calcium succinate composition is an effective binder of phosphorus in the gastrointestinal tract. The method comprises orally administering a quantity of a calcium succinate composition sufficient to bind with phosphorus in the GI tract and prevent its absorption. Preferably, a unit dose of the calcium succinate composition is between about 0.45 and 15 g calcium succinate (i.e., between about 3 and 100 millimoles of calcium as calcium succinate) and is administered in a pharmaceutically acceptable oral dosage form (i.e., a tablet, gelatin capsule, elixir, and so forth). In a most preferable embodiment of the present invention, the oral dose is ingested close in time with food and/or beverage consumption.

In addition, the present invention relates to a method of reducing serum phosphorus levels in a warm-blooded animal comprising treating the animal with a therapeutically effective amount of a phosphorus-binder composition comprising a calcium succinate composition.

The present invention also relates to a method of treating hyperphosphatemia in a warm-blooded animal comprising treating the animal with a therapeutically effective amount of a phosphorus-binder composition comprising a calcium succinate composition, thereby inhibiting uptake of phosphorus from the gastrointestinal tract.

Included within the scope of this invention is a method of treating hyperphosphatemia in a warm-blooded animal comprising administering pharmaceutical compositions comprising a calcium succinate composition and a suitable pharmaceutical carrier, thereby inhibiting the uptake of phosphorus from the gastrointestinal tract of the animal.

According to the method of the present invention, a phosphate binding composition comprising a calcium succinate composition that provides calcium in sufficient quantities to reduce phosphorus absorption from the gastrointestinal tract is administered orally, alone or preferably in combination with a stool softener. Other substances that are necessary to form a tablet or caplet as a delivery vehicle for the calcium succinate; in a hard gelatin capsule; together with a second phosphorus binder or other pharmaceutically useful substance) The calcium succinate is administered orally, preferably close in time to food and/or beverage consumption (i.e., concurrent with and/or within about 1 hour before or after ingestion of food or beverages).

The term “phosphorus,” in defining use of a calcium succinate composition as a phosphorus binder, is intended to embrace both inorganic and organic anions of phosphorus in the various forms that are capable of electrostatic reaction with a calcium ion, including, by way of example, phosphate (H₂PO₄¹⁻, HPO₄²⁻, and PO₄³⁻), pyrophosphate (P₂O₇⁴⁻), and the like.

By the term “calcium succinate composition” is meant calcium succinate (Chemical Abstracts Service Registry No. 140-99-8), a white amorphous powder containing approximately 25% calcium by weight. Calcium succinate has the molecular formula CaC₄H₄O₄ and a molecular weight of 156.15. Calcium succinate, which is also named butanedioic acid calcium salt or succinic acid calcium salt, is available commercially (e.g., Jost Chemical Co., St. Louis, Mo.). Also within the scope of this term are hydrates of calcium succinate, succinate salts containing both calcium and a second alkali metal ion (Na¹⁺, K¹⁺, or Li¹⁺) or alkaline earth metal ion (Mg²⁺), crystalline forms of calcium succinate, polymorphic forms of calcium succinate, calcium succinate having specific bulk densities or tap densities, and calcium succinate having specific particle sizes. Further included within the scope of this term are calcium succinate compositions coated with pharmaceutically acceptable materials intended to modify the release and/or bioavailability of calcium succinate (e.g., Eudragit, microcrystalline cellulose, hydroxypropylmethylcellulose phthalate, and so forth).

The term “calcium” means the calcium ion, Ca²⁺.

By a “stool softener” is meant any compound that makes a subject's stool softer and easier to pass. Conventional, efficacious and safe stool softeners include poly(ethylene glycol) having a molecular weight ranging from about 1,500 Daltons to about 10,000 Daltons, lactulose, lactitol, magnesium salts such as magnesium ascorbate and magnesium citrate, and cyclodextrins such as alpha-, beta-, and gamma-cyclodextrin and pharmaceutically acceptable beta-cyclodextrin derivatives.

The term “excipient material” is intended to mean any compound forming a part of the formulation which is not intended to have biological activity itself and which is added to a formulation to provide specific characteristics to the dosage form, including by way of example, providing protection to the active ingredient from chemical degradation, facilitating release of a tablet or caplet from equipment in which it is formed, and so forth.

By the terms “treating” and “treatment” and the like are used herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein encompasses any treatment of a disease in a mammal, particularly a human and includes: (a) preventing the disease from occurring in a subject which may be predisposed to the disease but has not yet been diagnosed as having it; (b) inhibiting the disease or arresting its development; or (c) relieving the disease, causing regression of the disease and/or its symptoms or conditions.

The phrase “therapeutically effective” is intended to qualify the amount of calcium succinate for use in the orally administered therapy which will achieve the goal of reducing elevated serum phosphorus levels by reducing or inhibiting, for example, the absorption of phosphorus from ingesta in the gastrointestinal tract, while avoiding adverse side effects typically associated with metal-containing phosphorus binding agents.

For the purpose of this disclosure, a warm-blooded animal is a member of the animal kingdom which includes but is not limited to mammals and birds. The most preferred mammal of this invention is human.

Surprisingly, the inventor has discovered that a phosphate binder comprising a calcium succinate composition provides unexpectedly effective phosphorus binding action having distinct advantages over conventional phosphate binders used by a subject requiring treatment for hyperphosphatemia. While not wishing to be bound by any particular hypothesis or theory, the inventor believes that five factors support using a calcium succinate composition as a phosphorus binder: (1) The solubility and ionization in water of a calcium succinate composition. (2) Weight percent calcium in calcium succinate. (3) Ability of the calcium in a calcium succinate composition to form phosphate salts having reduced bioavailability for absorption from the GI tract. (4) Safety. (5) Low risk for side effects. These factors are discussed in greater detail below.

Solubility and ionization in water. Ionized calcium in solution is the reactive species in Pi binders. Therefore, good solubility and extensive ionization in water are important selection criteria. Calcium succinate has a solubility of 1.28 g/100 mL in water at room temperature. [Dean J A. Lange's Handbook of Chemistry. 15^{th} Ed. New York: McGraw-Hill, Inc. 1999.] Preliminary studies by the inventor have shown that its solubility increases by at least two-fold in simulated gastric fluid (0.1 N hydrochloric acid). The calculations provided in Example 1, Equation 11, show that calcium succinate is essentially completely ionized in aqueous solution. Thus, all of the calcium ion provided by calcium succinate is available for phosphorus binding.

Weight percent calcium in the salt. The dose of calcium salt that must be ingested is directly related to the weight percent of calcium that is present in the salt. Calcium comprises 25% of the mass of calcium succinate. This percentage calcium exceeds that of calcium acetate (23%) and other calcium carboxylates that have been used or studied as Pi binders in the past (Table 1, above). Thus, lower doses of calcium succinate provide phosphorus-binding activity equivalent to the activity of calcium acetate and other calcium carboxylates shown in Table 1.

Ability to form phosphate salts having reduced systemic bioavailability. In Example 1, the inventor presents theoretical calculations which indicate that ionized calcium from calcium succinate will react readily and nearly quantitatively with Pi in the GI tract to form insoluble and unabsorbable calcium phosphates by the calcium phosphate precipitation reactions that are known to reduce absorption of phosphorus from the gastrointestinal tract. In addition, Markovic, Fowler, and colleagues have studied bone formation in vivo and have reported that octacalcium phosphate carboxylates (OCPCs) are formed by a mechanism that applies specifically to succinate salts but not to acetate, carbonate, or citrate salts. [Marković M, Fowler B O, Brown W E. Chem Mater 1993; 5: 1401-1405. Marković M, Fowler B O, Brown W E. J Cryst Growth 1994; 135: 533-538.] The inventor has discovered that this mechanism comprises yet another reaction by which calcium succinate, but not calcium acetate, calcium carbonate, or calcium citrate, acts as a Pi binder.

The inventor has confirmed the results of theoretical calculations by studying the phosphorus binding activity of calcium succinate in the pH range from about pH 4 (the pH of the stomach when food is present) to about pH 8 (the pH of the large intestine). Using the experimental conditions presented in detail in Example, the inventor has determined the extent of phosphorus binding at each value of solution pH and has found that the extent of phosphorus binding ranges from xx % phosphorus bound at pH 4 to xy % phosphorus bound at pH 8.

Safety. Hyperphosphatemic subjects (i.e., subjects exhibiting abnormally elevated serum phosphorus levels) may ingest gram doses of Pi binders three times each day for many years. Thus, long-term safety is a critical factor in selecting a suitable Pi binder. Succinate is one of several dicarboxylic acids that are intermediates in the citric acid cycle. Therefore, calcium succinate is likely to be biocompatible. Succinate is tasteless and odorless, different from acetate and formate, which have pungent and objectionable odors. Acetate causes regurgitation of acetic acid (vinegar breath) as well as gastric and esophageal irritation following ingestion by a subject. Formate is reported in the Merck Index [The Merck Index, 13^{th} Edition, Rahway, N.J.] to be a cause of albuminuria or hematuria and a strong irritant; succinate is not.

Low risk of side effects. Since large doses of the commonly used calcium carbonate and calcium acetate salts are required with each meal for optimal effectiveness, their side effects—distaste during ingestion and the constipation that often ensues later—may produce poor medication compliance; therefore, Pi control may remain suboptimal for patients. Other possible side effects of calcium carbonate and calcium acetate include gas, bloating, and headache, and increase in severity with dose. Calcium carbonate is poorly soluble in slightly acidic or near-neutral solutions, and the fact that gastric acid secretion is often impaired in hyperphosphatemic patients may limit its dissolution and ionization [Gold C H, Morley J E, Viljoen M. et al. Nephron 1980; 25:92-95. Hardy P, Sechet A, Hottelart C. et al. Artif Organs 1988; 22: 569-573. Tan C C, Harden P N, Rodger R S. et al. Nephrol Dial Transplant 1996; 11:851-853.] In addition, improperly sourced or poorly formulated calcium carbonate tablets may fail to dissolve, further limiting calcium availability in unpredictable ways. Calcium acetate is poorly tolerated, and distaste, “acetic acid breath” (“vinegar breath”), and discomfort have been reported to reduce patient compliance. Hypercalcemia has been observed in CKD patients using both calcium acetate and calcium carbonate. [Meric F, Yap P, Bia M J. Am J Kidney Dis 1990; 16: 459-464.] Epidemiologic studies show strong independent correlations between risk of death and either hyperphosphatemia or a high calcium-phosphorus product (i.e., [Ca, mg/dL]×[P, mg/dL]).

By comparison to either calcium carbonate or calcium acetate, calcium succinate exhibits low risk for side effects. Calcium succinate does not cause gas, acid regurgitation, gastric and esophageal irritation, or other side effects caused by conventional calcium salts.

The inventor has also discovered a phosphorus binding composition comprising a first quantity of a calcium succinate composition and a second quantity of a stool softener. A unit dose of said phosphorus binding composition comprises a first quantity of a calcium succinate composition that provides between about 3 millimoles and about 200 millimoles calcium as calcium succinate and a second quantity of a stool softener sufficient to inhibit constipation but not sufficient to cause diarrhea. The stool softener is selected from the group consisting of poly(ethylene glycol) having a molecular weight between about 1,500 Daltons and about 10,000 Daltons, lactulose, lactitol, magnesium ascorbate, magnesium citrate, alpha-cyclodextrin, beta-cyclodextrin, gamma-cyclodextrin, and a pharmaceutically acceptable beta-cyclodextrin derivative. Based on the recommendations of clinicians, including those published in the article entitled “Recommendations on chronic constipation (including constipation associated with irritable bowel syndrome) treatment” [Canadian Journal of Gastroenterology, volume 21 (Supplement B), pages 3B-22B, 2007] and in the monograph entitled “Systematic review of the effectiveness of laxatives in the elderly” [Health Technology Assessment, volume 1, number 13, pages 1-53], phosphate binder compositions comprising a first quantity of calcium as calcium succinate and a second quantity of a stool softer selected from the group consisting of poly(ethylene glycol) having a molecular weight in the range from about 1,500 to about 10,000 Daltons, lactitol, alpha-cyclodextrin, and magnesium ascorbate are preferred embodiments of the invention. Data concerning each stool softener are available for the skilled practitioner to select a quantity that is sufficient to inhibit constipation but not sufficient to cause diarrhea.

The inventor has verified by the experiments described in Example x that effective phosphorous binding takes place when a phosphate binder comprising a calcium succinate composition reacts with phosphate in the presence of a stool softener selected from the group.

DOSAGE FORMS. The pharmaceutical compositions of this invention can be administered by any means that effects contact of the therapeutically active ingredients (i.e., active ingredients) with the site of action in the body of a warm-blooded animal. A most preferred administration is by the oral route (i.e., ingestion). The active ingredients can be administered by the oral route in solid dosage forms, such as tablets, capsules, and powders, or in liquid dosage forms, such as elixirs, syrups, and suspensions. The pharmaceutical composition is preferably made in the form of a dosage unit containing a particular amount of each active ingredient.

In general, the pharmaceutical compositions of this invention can be prepared by conventional techniques, as are described in Remington's Pharmaceutical Sciences, a standard reference in this field [Gennaro A R, Ed. Remington: The Science and Practice of Pharmacy. 20^{th} Edition. Baltimore: Lippincott, Williams & Williams, 2000]. For therapeutic purposes, the active components of this invention are ordinarily combined with one or more excipients appropriate to the indicated route of administration. Such capsules or tablets may contain a controlled-release formulation as may be provided in a dispersion of active compound in hydroxypropyl methylcellulose or related material known to alter the kinetics of release of the active agent. Solid dosage forms can be manufactured as sustained release products to provide for continuous release of medication over a period of hours using known pharmaceutical techniques. Compressed tablets can be sugar coated or film coated to mask any unpleasant taste and protect the tablet from the atmosphere, or enteric coated for selective disintegration in the gastrointestinal tract. Both the solid and liquid oral dosage forms can contain coloring and flavoring to increase patient acceptance.

Serum phosphorus levels rise easily after a large meal. Therefore, dosing for oral administration preferably comprises a regimen calling for administration of a therapeutic dose of calcium succinate close in time to the ingestion of food and/or beverages. Dosing may be subdivided in a manner in which a portion of the prescribed dose is ingested prior to consumption of food or beverages, another portion is ingested together with food or beverages, and yet other portions are ingested close in time after ingestion of food or beverages. Preferably, dosing occurs within about an hour prior to and after ingestion of food or beverages.

The following examples present hypothetically useful therapeutic applications of representative pharmaceutical compositions of the present invention and their anticipated outcomes in treating hyperphosphatemia in subjects requiring such treatment. The examples are representative of the scope of the invention, and as such are not to be considered or construed as limiting the invention recited in the appended claims.

### Example 1

Theoretical calculations of Pi binding as a measure of its ability to form phosphate salts. Phosphorus binding results from reaction of a metal cation with Pi to form an insoluble metal-phosphate salt. Equilibrium constant expressions for the chemical reactions involved in the interactions of Pi and calcium salts (Table 2) have been used to calculate theoretical binding of Pi by calcium succinate. Theoretical calculations are based on the chemical reaction in which inorganic phosphate (H₂PO₄¹⁻, HPO₄²⁻, and/or PO₄³⁻) that is in solution with a metal ion reacts to form insoluble metal phosphate salts.

In the discussion that follows, binding at equilibrium has been estimated by calculating the total amount of phosphate that could exist in a saturated solution of the binder cation-phosphate precipitate in the presence of the excess binder at the particular pH of interest. The binding reaction is the formation of the insoluble phosphate(s): aB+bP=BₐP_{b}(s) (where B=binder cation, P=PO₄³⁻ or HPO₄²⁻, s=solid or precipitate form, a=mol of B, b=mol of P). The concentration at equilibrium was assumed to be governed by the solubility product constant, Kₛₚ=[B]^{a}[P]^{b}, were [X] denotes molar concentration of component X in a saturated solution, and Kₛₚ is the solubility product constant for the reaction (Table 4). Total phosphate concentration was obtained by simultaneous solution of the solubility product constant expressions and the equilibrium constant expressions governing the relative amounts of inorganic phosphate species (H₃PO₄, H₂PO₄₋, HPO₄²⁻, PO₄³⁻). The possibility that the binder cation formed soluble complexes with other species in solution, such as succinate, was also considered in the system of equations solved for determining total phosphate. For these calculations, the effect of ionic strength was ignored, and activity coefficients were assumed to be unity. Percent binding was calculated as precipitated phosphate divided by total phosphate times 100.

TABLE 2
Equilibria and related equilibrium constants

| Equilibrium | Log K | Eq. No. |
|---|---|---|
| HPO₄²⁻ + H⁺ = H₂PO₄¹⁻ | 6.83 | (1) |
| PO₄³⁻ + H⁺ = HPO₄²⁻ | 11.72 | (2) |
| H(Succ)¹⁻ + H⁺ = H₂(Succ) | 3.92 | (3) |
| (Succ)²⁻ + H⁺ = H(Succ)¹⁻ | 5.29 | (4) |
| Ca²⁺ + H₂PO₄¹⁻ = CaH₂PO₄¹⁺ | 1.06 | (5) |
| Ca²⁺ + HPO₄²⁻ = CaHPO₄ | 1.90 | (6) |
| Ca²⁺ + H(Succ)¹⁻ = CaH(Succ)¹⁺ | 0.54 | (7) |
| Ca²⁺ + (Succ)²⁻ = Ca(Succ) | 1.25 | (8) |

The binding of phosphorus by calcium succinate at pH 5 will be taken as an example. If the initial total phosphate concentration is 0.0172 M (or 320 mg/600 mL) and that of Ca²⁺ is 0.0625 M (or 1,500 mg/600 mL, the equilibrium constant expressions (Table 2) governing the hydrogen-phosphate equilibria may be used to calculate the concentration of various forms of phosphate at pH 5. At pH 5, the dominant form of succinate is H(Succ)⁻¹. Therefore, the calcium-succinate equilibrium is defined by the following reactions and their solubility product constant expressions (K_{SP}):

Ca²⁺=(Succ)⁻²=Ca(Succ)(s) K_{SP}=9.77 (9)

Succ²⁻+H⁺=H(Succ)⁻¹ K_{SP}=1.45×10⁵ (10)

### Combining Equations 9 and 10:

Ca(Succ)(s)+H⁺=Ca²⁺+H(Succ)⁻¹ K_{SP}=1.48×10⁴ (11)

Note that K_{SP} is large, indicating that the equilibrium in Eq. 11 rests largely to the right. On this basis, it is chemically reasonable to assume that the calcium ion is fully available to bind with phosphate.

At pH 5, the dominant form of phosphate is H₂PO₄¹⁻. Using the concentrations above for the precipitation of CaHPO₄ and Ca₃(PO₄)₂, it may be shown that only CaHPO₄ would precipitate. The following reactions occur at equilibrium:

CaHPO₄(s)=Ca²⁺+HPO₄²⁻ K_{SP}=4×10⁻⁷ (12)

HPO₄²⁻+H⁺=H₂PO₄¹⁻ K_{SP}=1.47×10⁷ (13)

Combining Equations 12 and 13, we obtain:

CaHPO₄(s)+H⁺=Ca²⁺+H₂PO₄¹⁻ (14)

for which

$${K}_{SP} = \frac{[ {Ca}^{2 +} ] [ {H}_{2} {PO}_{4}^{1 -} ]}{[ {H}^{+} ]} = 5.9 \qquad ( 15 )$$

If K_{SP} for CaHPO₄ were zero, all of the phosphate (0.0172 M) would precipitate as CaHPO₄, and there would be (0.0625−0.0172) M of calcium in solution. Because Kₛₚ is not zero, an additional amount, x mol of calcium and phosphate, remains in solution. Hence, at equilibrium, [H₂PO₄¹⁻=x and [Ca²⁺]=(0.0625−0.0172)+x. Substituting these values in Equation 16 and solving: x=0.00127 M at pH 5. The concentration of phosphate in the precipitate=(0.0172−x)=(0.0172−0.00127)=0.0159 M, and the percent binding=(phosphate in precipitate/total phosphate)×100=(0.0159/0.0172)×100=92.4%. These calculations indicate that calcium succinate is an excellent Pi binder.

### Example 2

In vitro Assessment of Phosphate Binding by Calcium Succinate. Test Preparations: Solutions of the test article (calcium succinate) and control articles (calcium acetate) were prepared in deionized, purified water having 18 MS2 or greater resistance. The pH of each solution was adjusted to the desired value by the addition of concentrated hydrochloric acid or sodium hydroxide, as appropriate.

Tests and Assays: Calcium succinate was assayed as described in the U.S. Pharmacopeia by dissolving an accurately weighed sample in water containing hydrochloric acid, adding hydroxynapthol blue as an indicator, and titrating to a blue endpoint with edetate disodium solution. An HPLC method with conductivity detection was developed and validated for use in the determination of succinate, acetate and phosphate. The separation was performed on a Dionex AS11 Cation-Exchange HPLC column integrated with an Agilent Series 1100 HPLC system, and detection of the anionic species was enabled using a Dionex ED50 Electrochemical Detector, operating in Conductivity Mode. Limits of Detection and Quantitation were enhanced through the use of a Dionex Anion Self-Regenerating Suppressor. After assay-specific development and verification of assay performance were completed, the analysis of phosphate and succinate or acetate was performed by sampling the test solution and diluting it, if necessary, to a concentration within the linear range of the Assay. The sample was then injected onto the HPLC column and eluted with a sodium hydroxide gradient. Data were acquired using Agilent ChemStation® software.

Experimental Methods: Experiments were completed in triplicate in which 2.77 g of Na₂HPO₄.7 H₂O (equivalent to 320 mg of elemental phosphorus) was dissolved in 570 mL of deionized water. The test or control binder was dissolved in deionized water to a volume of 30 mL. The binder solution was added to the phosphorus solution to give a final volume of 600 mL. For each binder study, the phosphorus solutions were titrated by addition of dilute HCl or dilute NaOH to five different initial pH levels: 4, 5, 6, 7, and 8. (These solution pH's span the pH range of the gastrointestinal tract.) Then the beakers containing the solutions were covered with plastic wrap and placed on a stir plate that agitated the solution at ˜20 cycles per minute overnight. This stirring rate has been selected because in vitro antacid activity at such low stirring rates has been reported to correlate well with in vivo antacid activity in the stomach. Samples for ion chromatographic assay of solution phosphate (Pi) were taken at 24 hours post-mixing (the maximum time available for phosphorus binding that has been reported in related in vivo studies). The decrease in phosphorus concentration from the original concentration in the phosphorus solution to that of the filtrate represents the bound phosphorus.

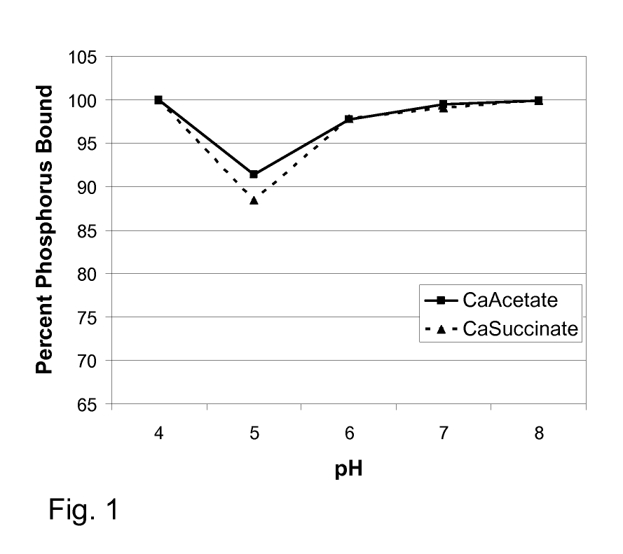

The experimental data (FIG. 1) demonstrate that Pi binding by calcium acetate, the U.S. standard of care, decreases from 100% at pH 4 to 92% at pH 5 and then increases to 100% as the pH increases to 6, 7, or 8. Pi binding by calcium succinate matches or exceeds the Pi binding activity of calcium acetate at each pH value in the range from pH 4 to 8.

Further, a comparison of the Pi binding exhibited by a calcium succinate composition of the invention with the binding observed for other calcium salts is provided in Table 3.

TABLE 3
Observed in vitro Pi binding for calcium compounds at pH 6

| | % Ca, | Observed in vitro Pi | | |
|---|---|---|---|---|
| Calcium | by | Binding, % | | |
| Source | weight | pH 4.0 | pH 6.0 | Comments |
| Calcium | 23% | 58.6% | 93.8% | Inventor's experiments |
| Acetate | | | | (Note 1); confirm data |
| | | | | of Sheikh et al. |
| | | | | (NOTE 2) |
| Calcium | 25% | 59.4% | 94.0% | Inventor's experiments |
| Succinate | | | | (Note 1) |
| Calcium | 40% | Not | 90 | NOTE 2 |
| Carbonate | | Reptd. | | |
| Calcium | 21% | Not | 10 | 20% at pH 6.5 (NOTE 2) |
| Citrate | | Reptd. | | |
| Calcium | 31% | Not | Not Reptd. | Not reported. |
| Formate | | Reptd. | | |
| Calcium | 14% | Not | 90 | NOTE 2 |
| Lactate | | Reptd. | | |
| Calcium | 9.3% | Not | 90 | NOTE 2 |
| Gluconate | | Reptd. | | |

NOTE 1:
Mean values of triplicate determinations of phosphate and acetate or succinate by anion-exchange HPLC with conductivity detection.
NOTE 2:
Sheikh M S, Maguire J A, Emmett M, Santa Ana C A, Nicar M J, Schiller L R, Fordtran J S. Reduction of dietary phosphorus absorption by phosphorus binders: A theoretical, in vitro, and in vivo study. J Clin Invest 1989; 83: 66-73.

The data in Table 3 confirm that Pi binding by both calcium acetate and calcium succinate occurs at values of pH as low as pH 4.0 but is nearly quantitative at values of pH near neutrality. Note as well that these in vitro data fail to predict the differences in Pi binding by the various calcium salts that are observed in vivo, nor do these in vitro data reveal the poor dissolution of calcium carbonate in the stomach and the “vinegar breath” associated with ingestion of calcium acetate. Neither of these shortcomings is observed when calcium succinate of the present invention is used as a Pi binder.

### Example 3

### Phosphate Binding by Calcium Succinate/Stool Softener Compositions

Experimental Methods Experiments were completed in 0.1 M borate buffer. Sodium phosphate dibasic heptahydrate (Na₂HPO₄.7 H₂O; 762.12 mg, 2.84 mmol; equivalent to 357 mg of phosphate) was dissolved in 100 mL of 0.1 M borate buffer. One milliliter of the resulting solution was removed and diluted volumetrically to 50 mL with 100 μM KOH solution for determination of the initial phosphate concentration. Calcium succinate monohydrate (681.95 mg, 3.9 mmol Ca) was added as the solid to the stirred phosphate solution to provide “Binding Solution 1.” The pH of the resulting solution was determined as 6.26. After Binding Solution 1 was stirred at ambient temperature for 24 hours, 1 mL of the supernatant was removed, filtered, transferred to a 50-mL volumetric flask, and diluted to volume with 100 μM KOH solution for determination of the final phosphate concentration in Binding Solution 1. (A period of 24 hr was selected, because this corresponds to the maximum time available for phosphorus binding that has been reported in related in vivo studies.)

In a second experiment, sodium phosphate dibasic heptahydrate (Na₂HPO₄.7 H₂O; 762.12 mg, 2.84 mmol; equivalent to 357 mg of phosphate) was dissolved in 100 mL of 0.1 M borate buffer. Poly(ethylene glycol) (PEG, average molecular weight 2,000 Daltons; 100 mg) was added, and the solution was stirred until dissolution was complete. One milliliter of the resulting solution was removed and diluted volumetrically to 50 mL with 100 μM KOH solution for determination of initial phosphate concentration. Calcium succinate monohydrate (682.23 mg, 3.9 mmol Ca) was added as the solid to the stirred phosphate/PEG solution to provide “Binding Solution 2” (a binding solution containing a stool softener). The pH of the resulting solution was determined as 6.28. After Binding Solution 2 was stirred for 24 hours, 1 mL of the supernatant was removed, filtered, transferred to a 50-mL volumetric flask, and diluted to volume with 100 μM KOH solution for determination of the final phosphate concentration in Binding Solution 2.

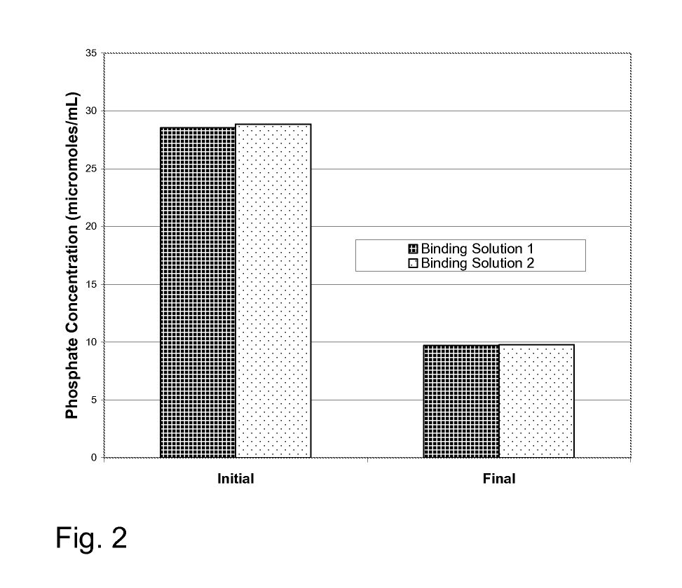

Samples were analyzed using a validated ion chromatographic assay of solution phosphate (Pi). The decrease in phosphorus concentration from the original concentration in the phosphorus solution to that of the filtrate represents the bound phosphorus. The experimental data (FIG. 2) demonstrate that Pi binding by a phosphate binder of the invention is identical in the absence (Binding Solution 1) or in the presence (Binding Solution 2) of a stool softener.

### Example 4

Calcium Succinate Composition as a Phosphate Binder in Humans. In vivo phosphorus binding by a calcium succinate composition of the invention and a placebo will be assessed in 10 healthy human subjects. The calcium succinate composition will be formulated to contain 169 mg of calcium as calcium succinate and 50 mg of poly(ethylene glycol) stool softener and will be presented as a gelcap. Each subject will be studied on three separate test days: fast, placebo, and calcium succinate composition. Net calcium absorption will be measured by a validated method, such as the one described in detail by Bo-Linn, G. W. et al., Journal of Clinical Investigation, 73: 640-647 (1984). The procedure that will be followed is described below.

On each day, subjects will be prepared by a mannitol-electrolyte gastrointestinal lavage, in order to cleanse the gastrointestinal tract. Four hours after completion of the washout, subjects will consume 25 mEq. of calcium (as calcium succinate) or a placebo (lactose) with 100 mL of deionized water. On one of the test days (the fast day), subjects will ingest no meal, placebo or calcium succinate; the rest of the procedure will be the same. Then each subject will eat a test meal of 80 g ground sirloin steak, 100 g French fried potatoes, 30 g Swiss cheese and 250 mL water containing 10 g of polyethylene glycol (PEG3500) as a non-absorbable marker. After the meal, each subject will consume 25 mEq of calcium, in the same form as will have been consumed prior to the meal, or additional placebo, with 100 mL of water. Duplicate meals will be prepared (one for consumption and one to be analyzed for calcium and phosphorus). The duplicate meals will be analyzed for calcium and phosphorus and are expected to contain about 350 mg of phosphorus and about 215 mg of calcium.

Calcium succinate composition will be administered in gelatin capsules. The total dose will equal 4.2 mmol of calcium (equivalent to 169 mg elemental calcium), one half of the dose taken just before the meal and the other half immediately after the meal. On one test day a placebo will be taken instead of calcium succinate. The order of testing will be randomized.

Ten hours after a meal, a second lavage will be begun, using the procedure described above. This will remove unabsorbed material from the gut. All urine voided during the 10-hour period will be collected and analyzed for phosphorus and calcium. Rectal effluent will be collected, pooled with any stool passed during the 10-hour period and analyzed for phosphorus and calcium. Absorption will be calculated according to the following equation:

Net phosphorus (P) absorption=(P content of duplicate meal, mg)−(Total Effluent P, mg)

Net calcium (Ca) absorption=(Ca content of meal, as determined from duplicate meal, mg)+(Ca ingested as Ca succinate, mg)−(Total Effluent Ca, mg)

The results are expected to demonstrate that calcium succinate results in the inhibition of phosphorus absorption, when ingested close in time to food and beverage consumption. In other words, it is anticipated that on the placebo day, as much as about 70% or more of the dietary phosphorus will be absorbed from the GI tract of each subject. By comparison, on the day in which calcium succinate is ingested close in time to food and beverage consumption, it is anticipated that as little as about 20% of dietary phosphorus will be absorbed. In addition, the results are expected to demonstrate that calcium succinate is an efficient inhibitor of calcium absorption, when ingested close in time to food and beverage consumption. In other words, it is anticipated that on the placebo day, about 20-40% of the dietary calcium will be absorbed from the GI tract of each subject. By comparison, on the day in which calcium succinate is ingested, it is anticipated that less than about 10% change will be observed in the percentage of the dietary calcium that is absorbed from the GI tract of each subject.

All mentioned references are incorporated by reference as if here written. When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

Without further elaboration, it is believed that one skilled in the art can, using the preceding description, utilize the present invention to its fullest extent. The following specific embodiments are, therefore, to be construed as merely illustrative, and not limitative of the remainder of the disclosure in any way whatsoever.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

I claim:

1. A method of inhibiting absorption of phosphorus-containing anions from the gastrointestinal tract of a subject comprising orally administering to the subject a phosphorus-containing anion-binding composition in a quantity sufficient to bind with said phosphorus-containing anions in the gastrointestinal tract of the subject, wherein said phosphorus-containing anion-binding composition comprises a calcium succinate composition.

2. The method according to claim 1 wherein the phosphorus-containing anion-binding composition provides a unit dose of calcium succinate of between 3 and 200 milliequivalents of calcium.

3. The method according to claim 1 wherein the phosphorus-containing anion-binding composition is in tablet form.

4. The method according to claim 1 wherein the phosphorus-containing anion-binding composition is in capsule form.

5. The method according to claim 1 wherein the phosphorus-containing anion-binding composition is in a liquid form.

6. The method according to claim 1 wherein the phosphorus-containing anion-binding composition is in a powder form.

7. A method of inhibiting absorption of phosphorus from the gastrointestinal tract of a subject comprising orally administering to the subject a phosphorus-containing anion-binding composition in a quantity sufficient to bind with phosphorus-containing anions in the gastrointestinal tract, wherein said phosphorus-containing anion-binding composition comprises a calcium succinate composition and the phosphorus-containing anion-binding composition is administered at a time close time to the time of consumption of food or beverages by the subject.

8. The method according to claim 6 wherein the calcium succinate composition is present in an amount sufficient to provide between 3 and 200 milliequivalents of calcium.

9. The method according to claim 6 wherein the phosphorus-containing anion-binding composition is in tablet form.

10. The method according to claim 6 wherein the phosphorus-containing anion-binding composition is in gelatin capsule form.

11. The method according to claim 6 wherein the phosphorus-containing anion-binding composition is in a liquid form.

12. The method according to claim 6 wherein the phosphorus-containing anion-binding composition is in a powder form.

13. A pharmaceutical composition useful for treating hyperphosphatemia in a warm-blooded animal comprising a phosphorus-containing anion-binding composition comprising a quantity of a calcium succinate composition sufficient to bind with phosphorus-containing anions in the gastrointestinal tract of the subject.

14. A method for reducing the serum phosphorus level in the blood of a subject comprising orally ingesting a phosphorus-containing anion-binding composition comprising a quantity of a calcium succinate composition sufficient to bind with phosphorus-containing anions in the gastrointestinal tract of the subject, thereby inhibiting phosphorus uptake into the systemic circulation of the subject.

15. A method of inhibiting absorption of phosphorus from the gastrointestinal tract of a subject and reducing the risk of side effects comprising orally administering to the subject at a time close time to the time of consumption of food or beverages by the subject a phosphorus-containing anion-binding composition comprising first quantity of a phosphorus-containing anion-binding composition of a calcium succinate composition sufficient to bind with phosphorus-containing anions in the gastrointestinal tract and a second quantity of a stool softener selected from the group consisting of poly(ethylene glycol) having a molecular weight between about 1,500 Daltons and about 10,000 Daltons, lactulose, lactitol, magnesium ascorbate, magnesium citrate, alpha-cyclodextrin, beta-cyclodextrin, gamma-cyclodextrin, and a pharmaceutically acceptable beta-cyclodextrin derivative.

16. The method according to claim 13 wherein the phosphorus-containing anion-binding composition provides a first quantity of a calcium succinate composition in an amount sufficient to provide between 3 and 200 milliequivalents of calcium and a second quantity of stool softener in an amount sufficient to inhibit constipation but not cause diarrhea.

17. The method according to claim 13 wherein the phosphorus-containing anion-binding composition is in tablet form.

18. The method according to claim 13 wherein the phosphorus-containing anion-binding composition is in capsule form.

19. The method according to claim 13 wherein the phosphorus-containing anion-binding composition is in powder form.
